(19) United States
(12) Patent Application Publication
(10) Pub. No.: US 2023/0082586 A1
(43) Pub. Date: Mar. 16, 2023

(54) MULTIPLE THICKNESS SAW BLADE FOR PRECISION ORTHOPEDIC CUTS

(71) Applicant: Treace Medical Concepts, Inc., Ponte Vedra, FL (US)

(72) Inventors: Madeline Lindemann, Palm Coast, FL (US); Paul Dayton, Ankeny, IA (US); Jason May, St. John's, FL (US); John Treace, Ponte Vedra Beach, FL (US); Bryan Wilcox, St. Augustine, FL (US)

(21) Appl. No.: 17/946,797

(22) Filed: Sep. 16, 2022

Related U.S. Application Data
(60) Provisional application No. 63/245,186, filed on Sep. 16, 2021.

(51) Int. Cl. A61B 17/14; B23D 61/00

(52) U.S. Cl. CPC A61B 17/14; B23D 61/006

(57) ABSTRACT

A saw blade for use in an orthopedic procedure may have multiple regions of different thickness across the length of the saw blade. The saw blade may include at least one region that is comparatively thicker. The saw blade may also include at least one region that is comparatively thinner and position closer to an end of the saw blade having cutting teeth. In some configurations, the thicker region of the saw blade is an intermediate region located between two comparatively thinner regions. The intermediate thicker region can interface with a slot of a cutting guide while the comparatively thinner regions can be sides for engaging with a driver and controlling the kerf width of a bone cut made using the saw blade, respectively.

### CROSS-REFERENCE

This application claims the benefit of U.S. Provisional Patent Application No. 63/245,186, filed Sep. 16, 2021, the entire contents of which are incorporated herein by reference.

### TECHNICAL FIELD

This disclosure relates to saw blades for cutting bone, including systems providing a saw blade and cutting guide sized relative to each other for precision cuts.

### BACKGROUND

A variety of different saw blade designs are used in orthopedic procedures. Different saw blade designs may be used depending on the specific anatomy being operated on, the manufacturer of the saw blade used in the procedure, and the preferences of the clinician performing the procedure. In either case, a surgical saw blade is typically mounted on a drive unit that can be grasp by the clinician performing the surgical procedure. The drive unit can be active by the clinician to induce relative motion of the saw blade, allowing the teeth of the saw blade to cut into one or more bones being operated on by the clinician.

While precision bone cuts are important for all orthopedic procedures, high-precision bone cuts are particularly important when operating on comparatively small bones. Example small bone procedures include those performed on the hand or foot, where the bones are comparatively small compared to other areas of the anatomy. Excess bone removal and/or misalignment in cut direction can have a disproportionally large impact on the efficacy of a procedure when performed on small bones as compared to larger bones.

### SUMMARY

In general, this disclosure is directed to a saw blade for cutting bone as well as systems and techniques utilizing the saw blade. In some examples, the saw blade is designed so that the saw blade has multiple regions of different thickness over the length of the saw blade. For example, the saw blade may have a comparatively thinner distal region and a comparatively thicker proximal region, when the distal region is positioned closer to the bone to be cut than the proximal region during the surgical procedure. Configuring the saw blade with a comparatively thicker region can increase the stiffness of the saw blade as compared to a saw blade of uniformly thinner thickness. This can reduce the amount of flexing exhibited by the saw blade during cutting, reducing the likelihood that the blade flexes off of a desired cut plane resulting in a skewed cut. By configuring a distal portion of the saw blade with a comparatively thinner region, the kerf width (width of bone material removed by the cutting process) may be reduced as compared to if the thickness of the entire saw blade is increased. This can minimize excess bone remove and/or bone shortening during the surgical procedure.

In some implementations, a system is provided that includes a saw blade and a cutting guide. The cutting guide can define one or more slots through which the saw blade can be inserted. In use, a slot of the cutting guide can be positioned over a bone at a location where the bone is to be cut. The saw blade can then be inserted through the slot to cut the underlying bone. The faces of the cutting guide can guide and/or bound movement of the saw blade as the saw blade is advanced into and/or through the underlying bone to be cut. This can help provide a more accurate and controlled bone cut than preforming the bone cut freehand.

The width of the cutting guide slot (e.g., the distance between opposed faces defining the slot) is sized larger than the thickness of the saw blade. This provides clearance for the saw blade to be inserted through the slot. In practice with existing systems, however, the excess space between one or both faces of the cutting guide slot and the thickness of the saw blade can allow the saw blade to tilt off-axis in the slot. As a result, even when using a cutting guide, the saw blade can angle off of a desired cut plane defined by the cutting guide slot, resulting in a skewed cut.

In accordance with some examples of the present disclosure, a cutting guide and saw blade system is provided that minimizes or eliminates relative off-axis tilt between the saw blade and the cutting plane define by the cut guide slot. As noted above, the saw blade may have a comparatively thinner distal region and a comparatively thicker proximal region. The thickness of the proximal region can be sized and positioned relative to the width and position of the cut guide slot. In use, the comparatively thicker proximal region of the saw blade may be sandwiched between the opposed faces of the cutting guide slot, e.g., as the saw blade is advanced into and/or through an underlying bone to be cut. As a result, the amount of excess space between one or both faces of the cut guide slot and the thickness of the saw blade is reduced, limiting the amount of relative movement or “play” between the saw blade and cutting guide slot during the surgical procedure. Further, by configuring a distal portion of the saw blade with a comparatively thinner region, excess bone removal is reduced as compared to if the entire thickness of the saw blade is increased.

A saw blade according to the disclosure can have two or more different thickness regions. In some configurations, the saw blade includes three thickness regions: a proximal thickness region, a distal thickness region, and an intermediate thickness region between the proximal and distal thickness regions. The intermediate thickness region may be thicker than both the proximal and distal thickness regions. The intermediate thickness region may be increased, e.g., as discussed above, to reduce the amount of blade flexing and/or relative movement of the saw blade within a cut slot. The distal region may have a reduced thickness to limit excess bone removal. The proximal region may have a reduced thickness to allow the saw blade to engage a powered driver that is operable to drive cutting motion of the saw blade.

A saw blade according to the disclosure can have a variety of different cutting teeth patterns and configurations. In some examples, the cutting teeth are arrayed in a straight line to define a flat or straight cutting profile. In other examples, the cutting teeth are arrayed in a curved line (e.g., concave, convex) to define a curved cutting profile. In addition, the cutting teeth may be arranged co-planar with the body of the saw blade or may be offset relative to the plane defined by the body of the saw blade. For example, the cutting teeth may be alternately offset from the plane defined by the body of the saw blade, which can provide offset gaps between adjacent teeth to help clear bone chips removed during cutting. In this latter configuration, the saw blade may define an additional cutting thickness region larger than the distal thickness of the saw blade.

In one example, a saw blade for cutting bone is described that includes a saw blade body having a length extending from a proximal end to a distal end and a thickness defined between a first side face and a second side face opposite the first side face. The example specifies that the proximal end of the saw blade body includes an engagement region configured to releasably couple the saw blade body to a driver, the distal end of the saw blade body includes a plurality of cutting teeth, and the saw blade body defines at least three thickness regions. The thickness regions include a proximal thickness region defining a proximal thickness, a distal thickness region defining a distal thickness, and an intermediate thickness region between the proximal thickness region and the distal thickness region, the intermediate thickness region defining an intermediate thickness. The example states that the intermediate thickness is larger than both the proximal thickness and the distal thickness.

In another example, a bone cutting system is described that includes a bone cutting guide and a saw blade. The bone cutting guide includes at least one cutting slot positionable over a bone to be cut, the cutting slot defining a width. The saw blade is configured for insertion through the cutting slot. The saw blade includes a saw blade body having a length extending from a proximal end to a distal end and a thickness defined between a first side face and a second side face opposite the first side face. The example specifies that the proximal end of the saw blade body includes an engagement region configured to releasably couple the saw blade body to a driver and the distal end of the saw blade body includes a plurality of cutting teeth. The example also specifies that the saw blade body includes a first thickness region defining a first thickness and a second thickness region defining a second thickness, the first thickness region being distal of the second thickness region, and the second thickness region being configured to be positioned co-planar with the cutting slot when the saw blade is inserted into the at least one cutting slot. The example also specifies that a ratio of the second thickness divided by the width of the cutting slot is at least 0.8, and the second thickness is larger than the first thickness.

In another example, a method is described that includes positioning a cutting slot of a bone cutting guide over a bone to be cut, the cutting slot defining a width. The example also involves inserting a distal end of a saw blade defining a plurality of cutting teeth through the cutting slot and advancing the saw blade further into the cutting slot as the plurality of cutting teeth cut the bone. The example specifies that the saw blade body includes a first thickness region defining a first thickness and a second thickness region defining a second thickness, and the second thickness is larger than the first thickness. The example states that advancing the saw blade further into the cutting slot involves advancing the first thickness region into the bone and positioning the second thickness region co-planar with the cutting slot.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front and side views, respectively, of an example saw blade according to the disclosure.

FIGS. 2A and 2B illustrate an example configuration of the saw blade of FIGS. 1A and 1B having two thickness regions.

FIG. 3 is a perspective view of an example configuration of the saw blade of FIGS. 1A and 1B having a variable thickness across the width of the saw blade.

FIGS. 4A and 4B are side cutaway and bottom cutaway views, respectively, of an example cutting tooth configuration.

FIGS. 5A and 5B illustrate the saw blade of FIGS. 1A and 1B prior to and after being engaged with an example driver engagement hub, respectively.

FIG. 6 is a side view of an example driver to which a saw blade can be releasably attached via a driver attachment.

FIG. 7 is a perspective illustration of an example bone cutting system that includes a saw blade and a cut guide.

FIG. 8 is a side view of a cut guide with a saw blade of uniform thickness positioned through the cutting slot of the cut guide.

FIG. 9 is a perspective illustration of a foot showing an example cut guide arrangement that be used with a saw blade.

### DETAILED DESCRIPTION

This disclosure relates to saw blades used in orthopedic bone cutting procedures as well as systems and techniques utilizing such saw blades. In general, a saw blade according to the disclosure is characterized by having multiple regions of different thickness across the length of the saw blade. The saw blade may include at least one region that is comparatively thicker. The saw blade may also include at least one region that is comparatively thinner and position closer to an end of the saw blade having cutting teeth (optionally including the region defining the cutting teeth). In some configurations, the thicker region of the saw blade is an intermediate region located between two comparatively thinner regions. The intermediate thicker region can interface with a slot of a cutting guide while the comparatively thinner regions can be sides for engaging with a driver and controlling the kerf width of a bone cut made using the saw blade, respectively.

When used with a cutting guide (also referred to as a cut guide herein), the region of the saw blade having increased thickness can allow for a tighter interface between the blade and the slot(s) of the cutting guide. The tighter interface can enable the blade to slide relatively freely through the cutting guide slots while minimizing toggle of the blade within the slot. Increasing the thickness of the blade where the blade interfaces with the cut guide slot can reduce lateral bending of the blade while cutting. This can help the blade remain in line with the slot, resulting in more consistent and reproducible cuts between different users of the system and repeated uses of the system by the same user. Additionally or alternatively, higher blade rigidity can allow for the cutting of thin bone slices because the blade has less of a tendency to skive across the surface of the bone and into an adjacent bone portion and/or joint space.

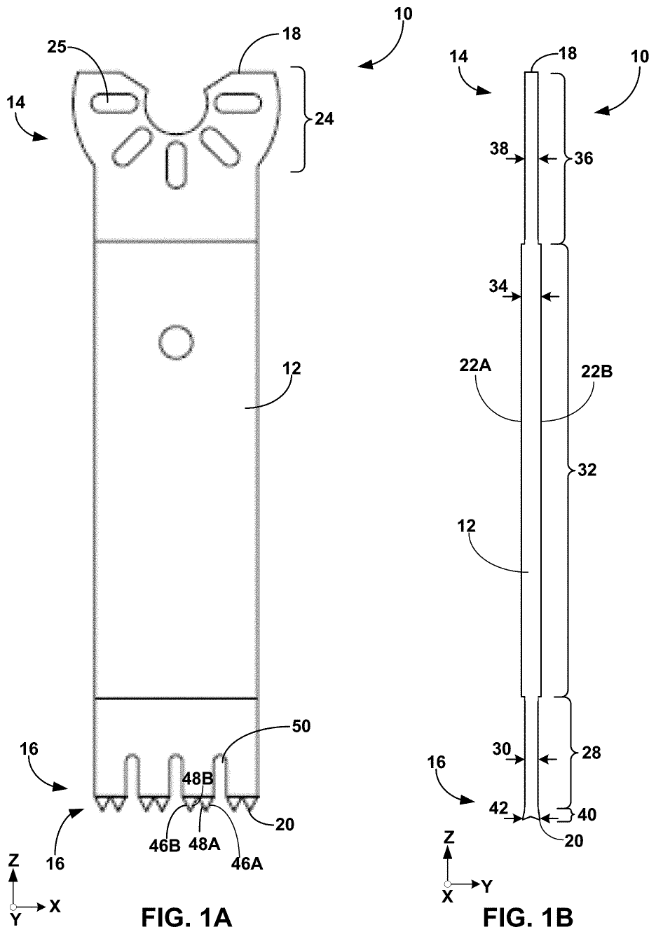

FIGS. 1A and 1B (collectively referred to as FIG. 1) are front and side views, respectively, of an example saw blade 10 according to the disclosure. Saw blade 10 is configured for use in cutting mammalian (e.g., human) bone during an orthopedic surgical procedure. While saw blade 10 can be used in any desired orthopedic application, the saw blade may be particularly useful for cutting comparatively small bones, such as those found in the hand and foot. In these procedures, high precision cuts are particularly beneficial because minor cutting inaccuracies result in comparatively large defects compared to the small size of the bone being cut.

As shown in FIG. 1, saw blade 10 is defined by a saw blade body 12 having a proximal end 14 and a distal end 16. Saw blade body 12 defines a length extending from a proximal-most end 18 to a distal-most end 20 (in the Z-direction indicated on FIG. 1A) and a width extending in a perpendicular crosswise direction (in the X-direction indicated on FIG. 1A) to the length. Saw blade body 12 has a first side face 22A and a second side face 22B opposite the first side face, with a thickness being defined between the two side faces (in the Y-direction indicated on FIG. 1B). The thickness of the saw blade body extends perpendicular to the length and width of the saw blade body and is smaller than both the length and the width.

In use, saw blade 10 can be attached to a driver that can drive movement of the saw blade relative to a bone to be cut, thereby providing a cutting motion. To attach saw blade body 12 to a driver, proximal end 14 of the saw blade body can include an engagement region 24. Engagement region 24 can be configured (e.g., shaped, patterned) to releasably couple the saw blade body to a driver. For example, engagement region 24 may have a shape profile and/or one or more through holes 25 configured for attachment to a hub of a driver. Different driver manufacturers can have different hub or other attachment configurations, and engagement region 24 of saw blade body 12 can be adapted to engage with any driver configuration.

Saw blade body 12 includes a plurality of cutting teeth 26 on the distal end 16 of the saw blade body. Cutting teeth 26 can have a variety of configurations, including those discussed in more detail below, and are designed to cut bone. Cutting teeth 26 can be arrayed across the width of saw blade body 12 at the distal end 16 of the saw blade body. Each cutting tooth may terminate in an apex or point that defines the distal-most end 20 of the saw blade body.

As briefly introduced above, saw blade body 12 is designed with multiple regions of different thickness. For example, with reference to FIG. 1B, saw blade body 12 may include at least two regions of different thickness, including a first thickness region 28 having a first thickness 30 and a second thickness region 32 having a second thickness 34. First thickness region 28 is located closer to the distal end 16 of saw blade body 12 (and may include the distal-most end 20 of the saw blade body) than the second thickness region. Similarly, second thickness region 32 is located closer to the proximal end 14 of saw blade body 12 (and may include the proximal-most end 18 of the saw blade body) than the first thickness region. In some configurations, saw blade body 12 has only two regions of different thickness across the entire length of the saw blade body. In other configurations, saw blade body 12 has more than two regions of different thickness across the length of the saw blade body (e.g., three, four, or more).

For example, in the illustrated configuration, saw blade body 12 is shown as further including a third thickness region 36 defining a third thickness 38. Third thickness region 36 is located proximally (e.g., farther away from the cutting teeth) of second thickness region 32 along the length of saw blade body 12. As a result, second thickness region 32 is located between (e.g., sandwiched between) first thickness region 28 and third thickness region 36. Accordingly, depending on the configuration of saw blade body 12, first thickness region 28 may also be referred to as a distal thickness region (with first thickness 30 being referred to as a distal thickness); second thickness region 32 may also be referred to as an intermediate thickness region (with second thickness 34 being referred to as an intermediate thickness); and third thickness region 36 may also be referred to as a proximal thickness region (with third thickness 38 being referred to as a proximal thickness).

Second thickness 34 of saw blade body 12 is larger (e.g., thicker, has a value of greater magnitude) than the first thickness 30 and, when saw blade body 12 is configured with a third thickness region, also third thickness 38. In some configurations, second thickness region 32 may be the thickest portion of saw blade body 12 across the entire length of the saw blade body.

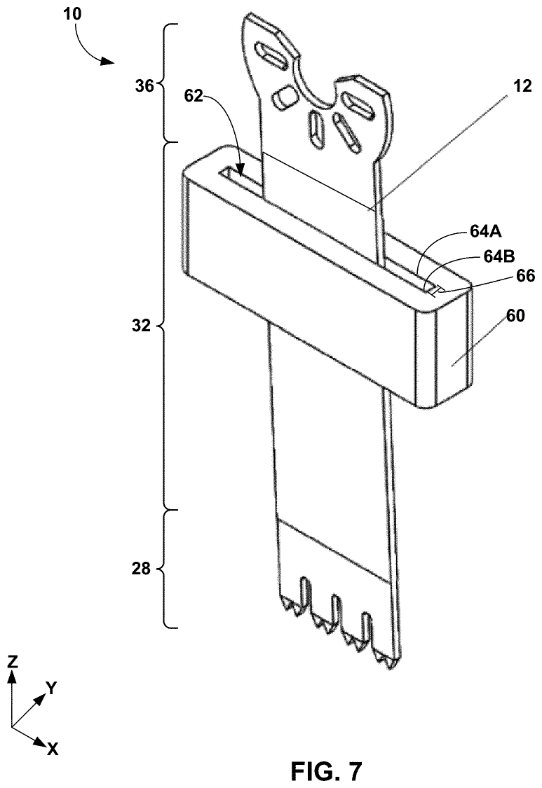

Configuring saw blade body 12 with a region 32 of increased thickness can be useful for a variety of reasons. Configuring the saw blade body with a comparatively thicker region can increase the stiffness of the saw blade as compared to a saw blade of uniformly thinner thickness. This can reduce the amount of flexing exhibited by the saw blade during cutting, reducing the likelihood that the blade flexes off of a desired cut plane resulting in a skewed cut. Further, as will be discussed in more detail in connection with FIG. 7, saw blade 10 may be used with a cut guide defining a slot through which the saw blade is inserted during a procedure. Configuring the saw blade body with a comparatively thicker region can limit angular movement of the saw blade within the slot, helping to provide more accurate and precise cuts. Further, configuring saw blade body 12 with a region 28 of comparatively reduce thickness can help minimize the kerf width (width of bone material removed by the cutting process). This can minimize excess bone remove and/or bone shortening during the surgical procedure.

In some examples, as discussed above, saw blade body 12 includes third thickness region 36 that is comparatively thinner than second thickness region 32 (in other words, third thickness 38 is smaller than second thickness 34). Configuring saw blade body 12 with a third, or proximal, thickness region 36 less than intermediate thickness region 32 can be beneficial to configure the saw blade body to mate with a variety of different driver designs. For example, a typical commercial orthopedic drivers may have a hub designed to receive a saw blade having a specific thickness. Increasing the overall thickness of saw blade 10 may render the saw blade incompatible with a particular orthopedic driver available to a clinician. Accordingly, configuring saw blade body 12 with third thickness region 36 that is comparatively thinner than second thickness region 32 (e.g., at least over the engagement region 24 of the saw blade) can provide the benefits of enhanced thickness while still allowing the saw blade to engage with a particular orthopedic driver available to a clinician. That said, in other configurations, an entire proximal portion from the end of first thickness region 28 to proximal-most end 18 may be comparatively thicker, and an appropriate orthopedic driver made available to mate with the comparatively thicker region of the saw blade.

In general, the first and second thickness regions (and, when included, optional third thickness region) of saw blade body 12 define the majority (e.g., entire length) of the saw blade body. In some implementations, however, saw blade body 12 may define an additional thickness region. For example, cutting teeth 26 of saw blade body 12 may be arranged coplanar with first thickness region 28 of saw blade body 12 or may be arranged out of plane with the first thickness region (e.g., to help provide space between adjacent cutting teeth for clearing bone fragments during cutting). When cutting teeth 26 are arranged coplanar with first thickness region 28, the cutting teeth may have a same (or lesser) thickness than first thickness 30. When cutting teeth 26 are arranged out of plane with the first thickness region 28, the cutting teeth may define an additional cutting tooth region 40 defining a cutting tooth thickness 42. When so configured, cutting tooth thickness 42 may be greater than first thickness 30.

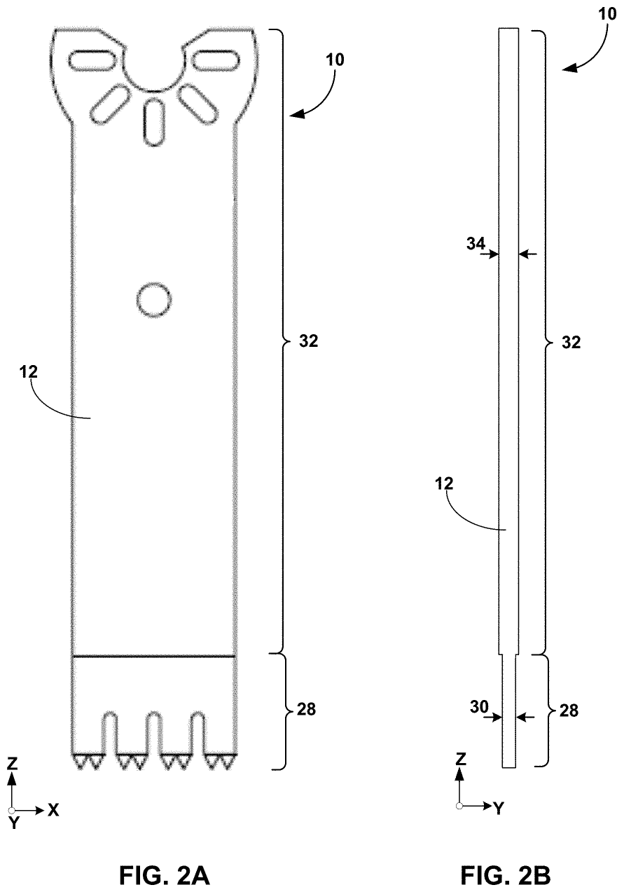

In various examples, saw blade body 12 may have only two regions of different thickness across the entire length of the saw blade body (from proximal-most end 18 to the distal-most end 20), only three regions of different thickness across the entire length of the saw blade body, or only four or more regions (e.g., four, five, etc.) of different thickness across the entire length of the saw blade body. For example, FIGS. 2A and 2B illustrate an example configuration of saw blade 10 having two thickness regions.

The relative lengths of the different thickness regions may vary, for example, based on the number of different thickness regions defined by saw blade body 12 and the overall length of the saw blade body. In various examples, second thickness region 32 extends over at least 30% of the length of saw blade body 12 (e.g., with the length be measured from proximal-most end 18 to distal-most end 20), such as at least 40%, at least 50%, at least 60%, at least 70%, or at least 80% of the length of the saw blade body. For example, second thickness region 32 may range from 40% to 90% of the length saw blade body 12, such as from 50% to 85% of the length of the saw blade body, or from 60% to 80% of the length of the saw blade body.

First thickness region 28 and third thickness region 36 (in configurations where the saw blade body includes a proximal third thickness region 36) each typically have a length less than length of second thickness region 32. First thickness region 28 and third thickness region 36 may each individually extend over less than 50% of the length of saw blade body 12, such as less than 40%, less than 30%, less than 20%, or less than 10%. For example, first thickness region 28 and third thickness region 36 may each individually range from 10% to 40% of the length of saw blade body 12, such as from 15% to 30% of the length of the saw blade body. First thickness region 28 may have a same length as third thickness region 36 or may have a different length than the third thickness region. Further, as discussed above, saw blade body 12 may not even include third thickness region 36.

When saw blade body 12 includes cutting teeth 26 that are positioned out of plane with first thickness region 28 to define a differentiated cutting tooth thickness region 40, the cutting tooth thickness region is typically a comparatively small portion of the overall length of saw blade body 12. For example, cutting tooth thickness region 40 may be less than 5% of the length of saw blade body 12, such as less than 3% of the length, or less than 2% of the length.

The relative thicknesses of the different thickness regions may also vary in different implementations of saw blade 10. In general, second thickness 34 may be comparatively thicker than first thickness 30 and third thickness 38 (when saw blade body 12 includes a proximal third thickness region). Second thickness 34 may be sufficiently thick to add flexural rigidity to saw blade 10 and/or help prevent angular shifting of the saw blade relative to a cut guide through which the saw blade is inserted (when used with a cut guide). In some implementations, a ratio of second thickness 34 divided by first thickness 30 (also referred to as a ratio of the intermediate thickness divided by the and distal thickness) and/or a ratio of second thickness 34 divided by third thickness 38 (also referred to as a ratio of the intermediate thickness divided by the proximal thickness) may each be at least 1.05, such as at least 1.1, at least 1.2, at least 1.3, or at least 1.5. For example, one or both ratios may be in a range from 1.05 to 2.0, such as from 1.1 to 1.5, or from 1.15 to 1.35.

When saw blade body 12 includes a cutting tooth thickness 42 that is different than distal first thickness 30, the cutting tooth thickness may be less than or substantially equal to second thickness 34. When used with a cut guide, configuring cutting tooth thickness 42 to be less than or substantially equal to (e.g., ±10%) second thickness 34 can help ensure that the cutting tooth thickness region 40 of saw blade body 12 passes through the cut guide slot with sufficient ease for the clinician using the cutting system. In other configurations, cutting tooth thickness 42 may be substantially a same thickness (e.g., ±10%) as first thickness 30 which, in turn, is thinner than second thickness 34, or may even be thinner than first thickness 30 (e.g., tapering to a point across the thickness of the saw blade body).

When saw blade body 12 is configured with both a distal first thickness region 28 and a proximal third thickness region 36 (with comparatively thicker intermediate second thickness region 32 there between), first thickness 30 and third thickness 38 may be the same as or different than each other. For example, first thickness 30 and third thickness 38 may each be the same thickness, or one of the two thicknesses may be thicker (larger) than the other thickness. In either configuration, second thickness 34 may still be larger than both the first thickness 30 and third thickness 38.

The absolute thicknesses of the different thickness regions of saw blade body 12 can vary depending on the characteristics of saw blade 10 and the intended orthopedic application utilizing the saw blade. In some examples, such as those involving small bone orthopedic procedures, second thickness 34 may be at least 0.55 mm, such as at least 0.58 mm, at least 0.60 mm, at least 0.62 mm, at least 0.65 mm, at least 0.7 mm, or at least 0.75 mm. For example, second thickness 34 may range from 0.55 mm to 1.0 mm, such as from 0.6 mm to 0.7 mm. First thickness 30 (and third thickness 38, when saw blade body 12 includes a thickness region) may each have a thickness less than 0.6 mm, such as less than 0.58 mm, less than 0.55 mm, or less than 0.51 mm.

In different examples, one or more thickness regions of saw blade body 12 (e.g., all thickness regions) may have a substantially constant or same thickness across the width of the saw blade body or may have a variable thickness across the width of the saw blade body. When configured with a substantially constant thickness across the width of saw blade body 12, the thickness may be substantially the same at each location within a given thickness region (e.g., each point across the width of a given thickness region) as each other location in the thickness region. When configured with a variable thickness across the width of saw blade body 12, a given thickness region may exhibit different thicknesses at different locations across the width of the thickness region. For example, one or more portions of a given thickness region may be comparatively thinner than one or more other portions of that thickness region. When so configured, the thickness of the given region may be considered, for purposes of defining the thickness of the region and comparing to other thickness regions, the maximum thickness across the width of the saw blade body in that region.

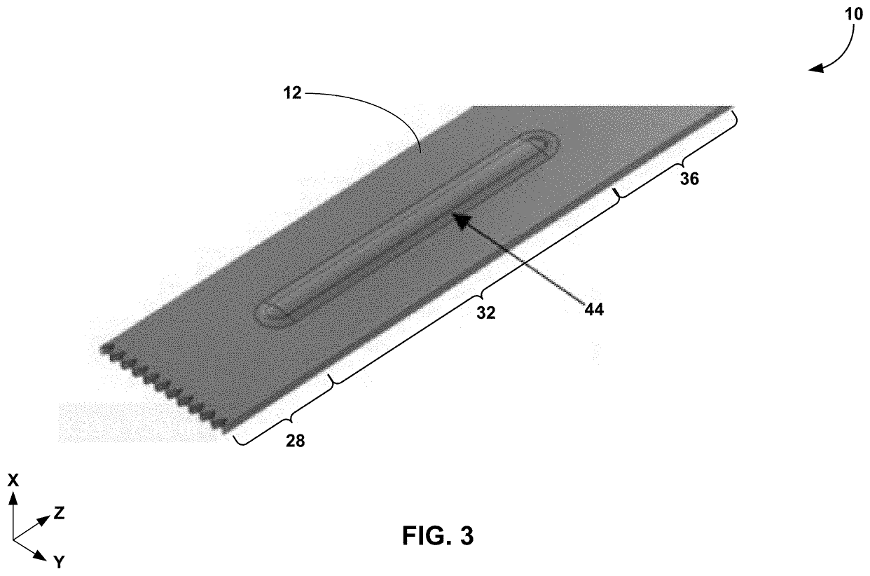

For example, FIG. 3 is a perspective view of an example configuration of saw blade 10 having a variable thickness across the width of the saw blade (in the Y-direction indicated on FIG. 3). In this example, saw blade body 12 includes a rib 44 extending outwardly from a remainder of the saw blade body to define a region of increased thickness. For example, saw blade body 12 may include one rib extending from a remainder of the first side face of the saw blade body and another rib extending from a remainder of the second side face of the saw blade body on the opposite side of the saw blade. Each rib may extend out the same distance albeit in opposite directions, or one rib may extend out a different direction than the other rib. Further, in other examples, saw blade body 12 may only have a single rib extending out from one side of the saw blade body (e.g., with the opposite side of the saw blade body being planar within the thickness region) and/or or may include a plurality of ribs extending out one or both sides of the saw blade body (e.g., multiple ribs arrayed across the width and/or length of the saw blade body). In either case, a rib or other structure defining increased thickness can push the face of the saw blade body outwardly relative to a remainder of the saw blade body in that region, thereby increasing the overall thickness of the saw blade body in that region. As noted, however, in other implementations, saw blade body 12 may have a substantially constant thickness across the width of the saw blade body rather than having a discontinuous or variable thickness as illustrated in the example of FIG. 3.

Any one of the thickness regions defined by saw blade body 12 can have a variable thickness across a width of the saw blade body (e.g., with the thickness of a particular thickness region be defined as the maximum thickness in that lengthwise region). In some examples, however, second thickness region 32 includes a variable thickness across a width of the saw blade body (e.g., being defined by one or more ribs and/or other relatively recessed and projecting features). In these examples, first thickness region 28 and/or third thickness region 36 (in configurations in which saw blade body 12 includes a third thickness region) may have a substantially constant thickness across an entire with of the saw blade body.

Independent of whether one or more thickness regions of saw blade body 12 have a constant or variable thickness across the width of the saw blade body, a variety of transition configurations can be provided between regions of different thickness. As one example, one thickness region may transition to another thickness region with a sharp step (e.g., 90° angle) between adjacent thickness regions. As another example, a tapered transition may be provided between one thickness region and an adjacent thickness region, e.g., such that the thickness of saw blade body 12 tapers from the thicker region to the thinner region. A tapered transition may be useful to help prevent the saw blade from catching on the edge of a cut guide slot as the saw blade is inserted into the cut guide slot.

With further reference to FIG. 1, the overall size of saw blade 10 may vary depending on the intended orthopedic application for the saw blade. In some implementations, saw blade body 12 has a length ranging from 20 cm to 150 cm, such as from 20 cm to 60 cm, or from 30 cm to 50 cm. The width of saw blade body 12 may range from 5 cm to 20 cm, such as from 5 cm to 15 cm, where the width is measured in the thickest region of the saw blade body (e.g., across second thickness region 32). In some implementations, saw blade body 12 has a length that is least twice the width of the saw blade body, such as at least three times the width of the saw blade body, at least four times the width of the saw blade body, or at least five times with the saw blade body.

Saw blade 10 includes a plurality of cutting teeth 26 on the distal or leading end of the saw blade that are configured to be advanced into a bone being cut during an orthopedic procedure. A variety of different cutting tooth designs can be used and implemented on saw blade 10. In some examples, each cutting tooth defines a generally triangular shape terminating in an apex. In the illustrated configuration of FIG. 1A, each cutting tooth 26 includes first and second rake surfaces 46A, 46B and first and second relief surfaces 48A, 48B. The first rake surface 46A and the first relief surface 48A intersect to define a first cutting edge (e.g., apex). The second rake surface 46B and the second relief surface 48B intersect to define a second cutting edge (e.g., apex). The two cutting edges are oppositely disposed from one another in this example configuration.

In some configurations, saw blade 10 includes one or more clean out opening 50 disposed between adjacent cutting teeth 26. Each clean out opening 50 can have a maximum width greater than the distance between the cutting edges of adjacent cutting teeth 26. In some examples, the clean out opening 50 may include an arcuate closed end and straight or angled side walls extending from the closed end. Between the straight or angled side walls, a substantially consistent or varying width is provided for each of the cutting teeth 26. In other words, a cutting tooth 26 may have a root portion disposed back of the cutting edge, and the root portion can have a substantially consistent or varying width moving from the root connection to the tip of cutting edge. Clean out opening 50 can help channel debris (e.g., cut bone) away from the cutting edge during a bone cutting procedure.

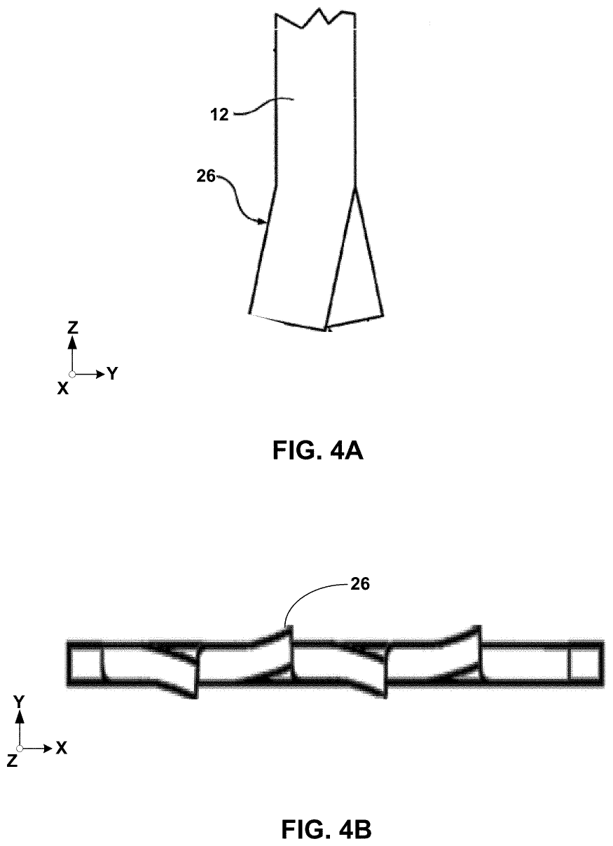

In some implementations, each cutting tooth of the plurality of cutting teeth 26 is positioned in the same plane as the distal thickness region 28. In other configurations, one or more teeth of the plurality of cutting teeth are positioned out of plane with distal thickness region 28. FIGS. 4A and 4B are side cutaway and bottom cutaway views, respectively, of cutting teeth 26 showing an example configuration where the cutting teeth are positioned out of plane. As shown in this example, each cutting tooth 26 is alternately offset from one another in opposite directions to provide cutting a groove wider than the thickness of the distal cutting region. In other words, one cutting tooth may extend from the cutting surface to one direction and another tooth extends to another direction. As illustrated, one cutting tooth 26 protrudes a distance from one side face of the cutting blade and the next adjacent cutting tooth protrudes the same distance from the other side face of the blade. In different configurations, groups of cutting teeth (e.g., two, three, or more) may protrude in a common direction followed by one or more other cutting teeth (e.g., two, three, or more) protruding in an opposite direction. Further, cutting teeth protruding in different directions may protrude the same distance from a centerline (albeit in different directions), or cutting teeth protruding in one direction may extend out a greater distance from the centerline then cutting teeth protruding in the opposite direction.

In the illustrated examples of saw blade 10, the plurality of cutting teeth 26 are illustrated as being arranged in a straight line. For example, each of the plurality of cutting teeth 26 are shown terminating at an apex, with the apexes being arranged in a linear line. In other examples, the plurality of cutting teeth may be arranged in a curved line (e.g., defining an outward concave or convex cutting surface), e.g., with the apexes of the plurality of cutting teeth are arranged in the curved line. Other shape profiles may be used without departing from the scope of disclosure.

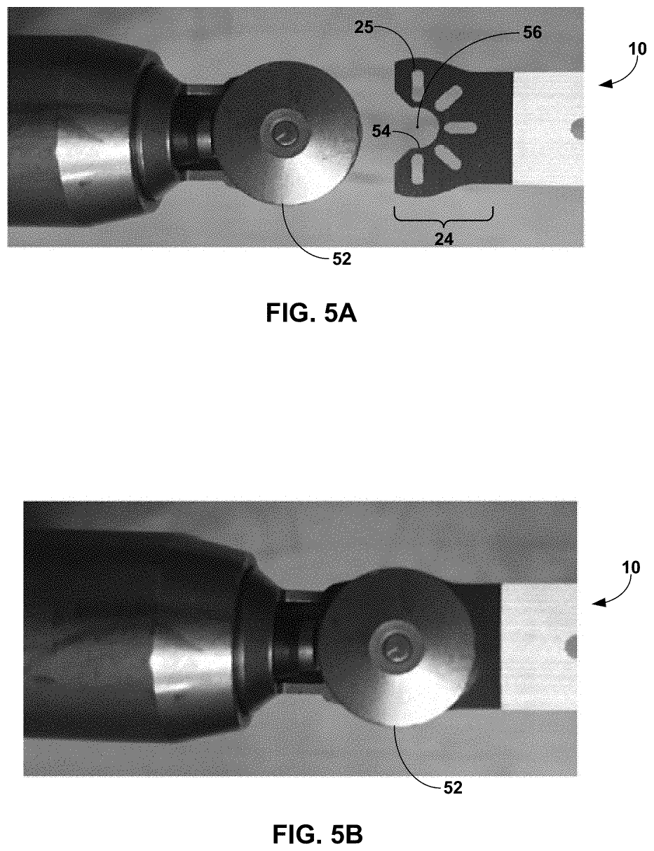

As discussed above, saw blade 10 can be configured with an engagement region 24 that allows the saw blade to releasably couple to a powered driver for driving movement of the saw blade. Engagement region 24 can have a variety of different shape and pattern configurations as appropriate to mate with a corresponding driver attachment (e.g., hub). FIGS. 5A and 5B illustrate saw blade 10 prior to and after being engaged with an example driver engagement hub 52, respectively. In some configurations, driver engagement region 24 includes is bulbous and includes a semi-circular slot 54 centered on an oscillation axis 56 that symmetrical with the longitudinal axis of the blade 10. Driver engagement region 24 may also include plurality of holes 25 to facilitate connection to a driver for driving engagement thereby.

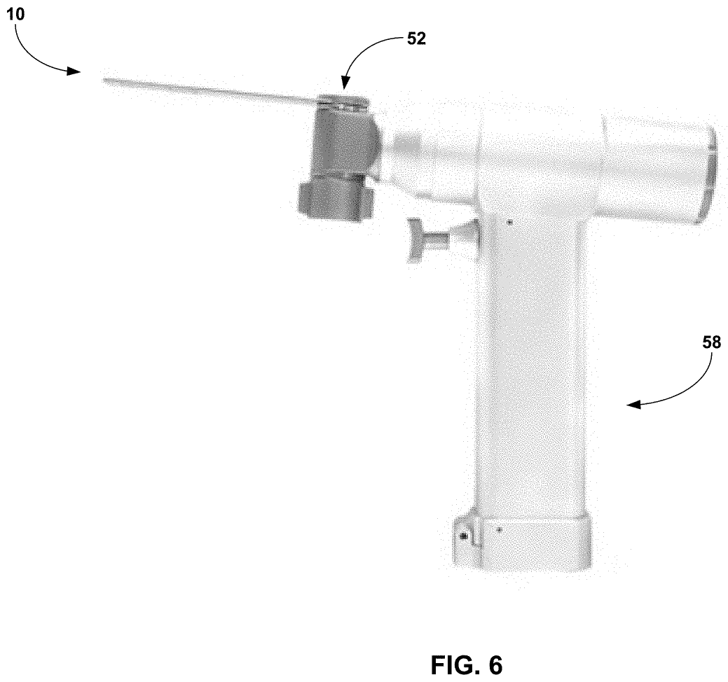

FIG. 6 is a side view of an example driver 58 to which saw blade 10 can be releasably attached via a driver attachment 52. Driver 58 may include a housing having an elongated, top-located barrel section and a pistol-grip shaped handle extending downwardly from the barrel section. A trigger can be located on the pistol-grip that is actuatable by the clinician to activate movement of the saw blade. A motor (e.g., DC motor, AC driver motor, pneumatically or hydraulically-driven motor) can be disposed inside the housing. In the illustrated version, driver 58 is a cordless power tool and houses a removable battery. In alternative versions, a power cord, an air line or a hydraulic line may be connected to the housing for providing the power needed to actuate the motor. Driver 58 can be configured as an oscillatory driver that oscillates saw blade 10 (e.g., side to side in the widthwise direction) or a reciprocating driver that reciprocates saw blade 10 (e.g., up and down in the lengthwise direction).

Saw blade 10 can be fabricated from any biocompatible materials, although most typically may be fabricated from metal (e.g., steel alloys, stainless steel, titanium, nickel alloys, combinations thereof). Saw blade 10 may or may not be fabricated from a single sheet of material. The single sheet of material may be cut or ground down in one or more regions to reduce the thickness in those regions and/or material may be deposited (e.g., solder deposited) along one or more regions to increase the thickness in those regions. Saw blade 10 is generally a unitary structure with no connected or moving features. For example, saw blade 10 may be a single continuous body (optionally formed of different sections of material permanently joined together) that do not move relative to each other. As a result, when saw blade 10 is attached to hub 52 of driver 58 and driven, the entirety of the saw blade moves and no one portion of the saw blade moves relative to any other portion of the saw blade.

Saw blade 10 having a region of comparatively larger thickness can be used freehand in a surgical procedure (e.g., in which the clinician plunges the blade into bone without the blade being guided by an additional guide structure) or may be used with a guide structure. For example, saw blade 10 may be used with one or more cut guides that are configured (e.g., sized and/or shaped) to guide cutting of one or more particular bones. A cut guide may be held or secured over the one or more bones to be cut and saw blade 10 guided by the cut guide to cut the underlying bones. Use of a cut guide can help the clinician more accurately and reliably cut the underlying bone.

Even when using a cut guide, however, relative movement of the saw blade within a slot of the cut guide may occur. FIG. 7 is a perspective illustration of an example bone cutting system that includes a saw blade 10 and an example cut guide 60. Cut guide 60 can define at least one cutting slot 62 through which saw blade 10 can be inserted (e.g., one, two, three or more cutting slots). In use, cut guide 60 can be positioned over a bone to be cut, and saw blade 10 advanced (e.g., downwardly) through the cut guide and into and/or through the underlying bone.

Cut guide 60 can have a first guide surface 64A and a second guide surface 64B separated from each other a distance to define cutting slot 62 having a width 66 (in the Y-direction indicated on FIG. 7). Saw blade 10 and cut guide 60 (in particular, width 66 of cutting slot 62) can be sized relative to each other to limit “play” or angular shifting of the saw blade within the cutting slot. For example, the increased thickness of second thickness region 32 can help fill out the space between the guide surfaces defining the cutting slot, limiting the amount of excess space and corresponding movement allowable between the saw blade and cut guide. This can help prevent the saw blade from tilting off a target longitudinal cutting axis within the cutting slot, which may otherwise cause bone and/or cartilage skiving.

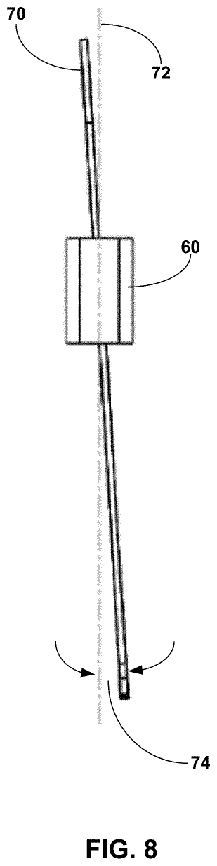

For example, FIG. 8 is a side view of cut guide 60 with a saw blade 70 of uniform thickness positioned through the cutting slot of the cut guide. In this example, a target longitudinal cutting axis 72 is defined through the cutting slot of the cut guide. Saw blade 70 is angularly offset from the target cutting axis 72 by an offset angle 74. This represents an error or miss cut relative to the target cutting axis.

In accordance with examples of the present disclosure, however, angular shifting of saw blade 10 within cutting slot 62 may be reduced or eliminated. The thickness of second thickness region 32 may be sized relative to the width 66 of cutting slot 62 (FIG. 7) to help reduce or eliminate angular shifting of the saw blade during a cutting operation. In some configurations, the thickness of second thickness region 32 may be sized relative to the width 66 of cutting slot 62 to limit angular offset 74 to 15° or less, such as 10° or less, 5° or less, 3° or less, or 2° or less.

To limit relative movement between saw blade 10 and cut guide 60, the thickness of the saw blade may be sized relative to the width of the cut guide slot. Second thickness 34 may be sized sufficiently close to width 66 of cutting slot 62 such that there is limited relative movement between the saw blade and cut guide when the saw blade is inserted into the cutting slot (e.g., when second thickness region 32 is positioned coplanar with the cutting slot). However, second thickness 34 may also be sufficiently smaller than width 66 of cutting slot 62 to allow saw blade 10 to pass through cutting slot 62 was sufficient ease.

In various implementations, a ratio of second thickness 34 divided by width 66 is at least 0.8, such as at least 0.82, at least 0.84, at least 0.86, at least 0.88, at least 0.9, at least 0.92, or at least 0.94. For example, the ratio of second thickness 34 divided by width 66 may range from 0.8 to 0.98, such as from 0.80 to 0.90, or from 0.82 to 0.88. The absolute size of cutting slot 62 may vary depending on the procedure for which cut guide 60 is intended to be used. In some examples, cutting slot 62 has a width 66 of at least 0.65 mm, such as from 0.65 mm to 1.0 mm, from 0.65 mm to 0.8 mm, or from 0.65 mm to 0.75 mm.

The second thickness region 32 of saw blade 10 having increased thickness may be located along the length of the saw blade at a position where, when the saw blade is cutting into an underlying bone, the second thickness region interfaces with and/or is co-planar with cutting slot 62 (e.g., the second thickness region is sandwich between first guide surface 64A and second guide surface 64B). In practice, second thickness region 32 may be partially or fully positioned in cutting slot 62 when cutting teeth 26 first contact the surface of the underlying bone to be cut or, alternatively, may not enter cutting slot 62 until cutting teeth 26 begin cutting the underlying bone thereby allowing the saw blade to advance deeper into the cutting slot (e.g., when the cutting teeth have partially but not fully cut into the underlying bone).

The saw blades and cutting systems of the present disclosure can be used in any desired orthopedic procedure and to cut any desired bones. In exemplary applications, the saw blades and/or cutting systems can be used during a surgical procedure performed on one or more bones, such as a bone alignment, osteotomy, fusion procedure, fracture repair, and/or other procedures where one or more bones are to be set in a desired position. Such a procedure can be performed, for example, on bones (e.g., adjacent bones separated by a joint or different portions of a single bone) in the foot or hand, where bones are relatively small compared to bones in other parts of the human anatomy. In one example, a procedure utilizing embodiments of the disclosure can be performed during a procedure to correct an alignment between a metatarsal (e.g. a first metatarsal) and a cuneiform (e.g., a medial cuneiform), such as a bunion correction. An example of such a procedure is a lapidus procedure. In another example, the procedure can be performed by modifying an alignment of a metatarsal (e.g. a first metatarsal). An example of such a procedure is a basilar metatarsal osteotomy procedure.

In various examples a surgical procedure performed using a saw blade and/or cutting system according to the disclosure may cut a comparatively small bone in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof. For example, a surgical technique may involve cutting an end of one bone and an end of an opposing bone separated from each other by a joint. For example, a surgical procedure may involve cutting and removing a portion of a metatarsal and cutting and removing a portion of cuneiform/cuboid separated from the metatarsal by a tarsometatarsal (“TMT”) joint. As another example, a surgical procedure may involve cutting and removing a portion of a metatarsal and cutting and removing a portion of proximal phalanx from the metatarsal by a metatarsophalangeal (“MTP”) joint. In either case, the amount of bone removed by cutting off the end of the bone may be comparatively small, such as less than 15 mm, less than 10 mm, less than 5 mm, less than 3 mm, less than 2 mm, or less than 1 mm. Small errors in cutting angle may be particularly impactful when removing comparatively small sections of bone. Details on example bone realignment instruments and techniques that can be used in conjunction with the present disclosure are described in U.S. Pat. No. 9,622,805, issued Apr. 18, 2017 and entitled “BONE POSITIONING AND PREPARING GUIDE SYSTEMS AND METHODS” the entire contents of which are incorporated herein by reference.

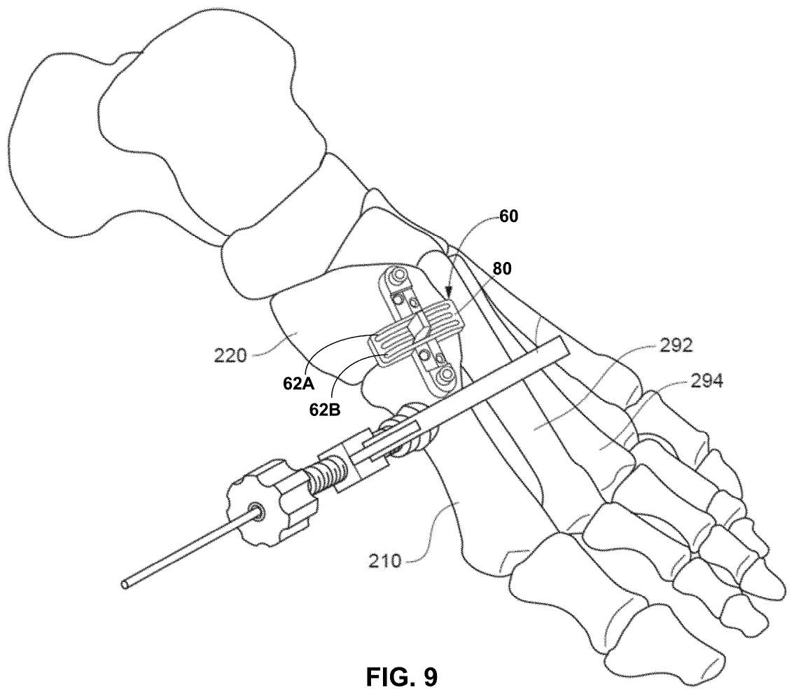

FIG. 9 is a perspective illustration of a foot showing an example cut guide arrangement that be used with saw blade 10. In this example, cut guide 60 is shown having a first cutting slot 62A positioned over an end of a medial cuneiform 220 to be cut and a second cutting slot 62B positioned over an end of a first metatarsal 210 to be cut. The first and second cutting slots 62A, 62B are separated from each other by a distance 80. The first metatarsal 210 is the medial most metatarsal and is medial of the second metatarsal 292 and third metatarsal 294. During a procedure, the clinician can insert saw blade 10 through cutting slots 62A and 62B to cut off an end of medial cuneiform 220 and first metatarsal 210, respectively. The clinician can cut either bone before the other bone and may perform additional optional realignment steps during the procedure. Cut guide 60 in the example of FIG. 9 is merely one example of a multi-slot cut guide, and other single or multiple-slot cut guides can be used with a saw blade according to the disclosure.

Various examples have been described. These and other examples are within the scope of the following claims.

1. A saw blade for cutting bone, the saw blade comprising:
a saw blade body having a length extending from a proximal end to a distal end and a thickness defined between a first side face and a second side face opposite the first side face;
the proximal end of the saw blade body comprising an engagement region configured to releasably couple the saw blade body to a driver;
the distal end of the saw blade body comprising a plurality of cutting teeth; and
the saw blade body defining at least three thickness regions, including:
a proximal thickness region defining a proximal thickness;
a distal thickness region defining a distal thickness; and
an intermediate thickness region between the proximal thickness region and the distal thickness region, the intermediate thickness region defining an intermediate thickness,
wherein the intermediate thickness is larger than both the proximal thickness and the distal thickness.

2. The saw blade of claim 1, wherein a ratio of the intermediate thickness divided by the proximal thickness and a ratio of the intermediate thickness divided by the distal thickness are each at least 1.1.

3. The saw blade of claim 1, wherein a ratio of the intermediate thickness divided by the proximal thickness and a ratio of the intermediate thickness divided by the distal thickness each range from 1.05 to 2.0.

4. The saw blade of claim 1, wherein the intermediate thickness region comprises at least half of the length of the saw blade body.

5. The saw blade of claim 1, wherein:
the proximal thickness region defines a length from the proximal end of the saw blade body to a proximal end of the intermediate thickness region;
the intermediate thickness region defines a length from a distal end of the proximal thickness region to a proximal end of distal thickness region;
the distal thickness region defines a length from a distal end of the intermediate thickness region to the plurality of cutting teeth; and
the length of the proximal thickness region and the length of the distal thickness region are each at least 20% of the length of the saw blade body.

6. The saw blade of claim 1, wherein the saw blade body comprises a cutting thickness region distal of the distal thickness region, the cutting thickness region defining a cutting thickness less than or equal to the intermediate thickness.

7. The saw blade of claim 6, wherein the cutting thickness region is substantially a same thickness as the distal thickness.

8. The saw blade of claim 6, wherein the cutting thickness region comprises less than 5% of the length of the saw blade body.

9. The saw blade of claim 1, wherein the saw blade body defines a unitary body with no connected features.

10. The saw blade body of claim 1, wherein the engagement region is configured to releasably couple the saw blade body to a drive member of at least one of an oscillatory driver and a reciprocating driver.

11. The saw blade of claim 1, wherein each of the at least three thickness regions has a uniform thickness across a width of the saw blade body.

12. The saw blade of claim 1, wherein the intermediate thickness region has a variable thickness across a width of the saw blade body, the intermediate thickness being a maximum thickness across the width of the saw blade body.

13. The saw blade of claim 12, wherein the intermediate thickness region comprises at least one rib extending outwardly relative to a remainder of the saw blade body, the at least one rib defining an increased thickness relative to the remainder of the saw blade body.

14. The saw blade of claim 12, wherein the proximal thickness region and the distal thickness region each have a uniform thickness across a width of the saw blade body.

15. The saw blade of claim 1, wherein the proximal thickness and the distal thickness are each less than 0.55 mm, and the intermediate thickness is at least 0.55 mm.

16. The saw blade of claim 1, wherein the proximal thickness and the distal thickness are each a same thickness.

17. The saw blade of claim 1, wherein the plurality of cutting teeth are alternately offset from one another in opposite directions to define a cutting thickness greater than the distal thickness.

18. A bone cutting system comprising:
a bone cutting guide comprising at least one cutting slot positionable over a bone to be cut, the at least one cutting slot defining a width;
a saw blade for insertion through the at least one cutting slot, the saw blade comprising:
a saw blade body having a length extending from a proximal end to a distal end and a thickness defined between a first side face and a second side face opposite the first side face;
the proximal end of the saw blade body comprising an engagement region configured to releasably couple the saw blade body to a driver; and
the distal end of the saw blade body comprising a plurality of cutting teeth;
the saw blade body comprising a first thickness region defining a first thickness and a second thickness region defining a second thickness, the first thickness region being distal of the second thickness region, and the second thickness region configured to be positioned co-planar with the at least one cutting slot when the saw blade is inserted into the at least one cutting slot;
wherein a ratio of the second thickness divided by the width of the at least one cutting slot is at least 0.8, and the second thickness is larger than the first thickness.

19. The system of claim 18, wherein the ratio ranges from 0.8 to 0.95, preferably from 0.85 to 0.92.

20. The system of claim 18, wherein a ratio of the second thickness divided by the first thickness is within a range from 1.05 to 2.0.

21. The system of claim 18, wherein:
the first thickness is less than 0.55 mm;
the second thickness is at least 0.55 mm; and
the width is at least 0.65 mm.

22. The system of claim 18, wherein the at least one cutting slot is configured to be positioned over at least one of a metatarsal and a cuneiform.

23. The system of claim 18, wherein the at least one cutting slot comprises a first cutting slot configured to be positioned over a metatarsal and a second cutting slot configured to be positioned over a cuneiform separated from the metatarsal by a tarsometatarsal joint, the first cutting slot and the second cutting slot each having a same width.

24. The system of claim 18, wherein the first thickness region ranges from 10% to 40% of the length of the saw blade body, and the second thickness region ranges from 60% to 80% of the length of the saw blade body.

25. The system of claim 18, wherein the plurality of cutting teeth are alternately offset from one another in opposite directions to define a cutting thickness greater than the first thickness and less than or equal to the second thickness.

26. The system of claim 18, wherein:
the saw blade body further comprises a third thickness region defining a third thickness;
the third thickness region is proximal of the second thickness region and configured to be positioned outside of the at least one cutting slot when the saw blade is inserted into the at least one cutting slot; and
the second thickness is larger than the third thickness.

27. The system of claim 26, wherein the first thickness and the third thickness are substantially a same thickness.

28. The system of claim 18, wherein:
the plurality of cutting teeth each terminate at an apex;
the apex of each of the plurality of cutting teeth are arranged in a linear line; and
a cleanout opening is disposed between adjacent teeth.

29. The system of claim 18, wherein the engagement region is configured to releasably couple the saw blade body to a drive member of at least one of an oscillatory driver and a reciprocating driver.

30. A method comprising:
positioning a cutting slot of a bone cutting guide over a bone to be cut, the cutting slot defining a width;
inserting a distal end of a saw blade defining a plurality of cutting teeth through the cutting slot; and
advancing the saw blade further into the cutting slot as the plurality of cutting teeth cut the bone,
wherein the saw blade body comprises a first thickness region defining a first thickness and a second thickness region defining a second thickness, the second thickness is larger than the first thickness, and advancing the saw blade further into the cutting slot comprises advancing the first thickness region into the bone and positioning the second thickness region co-planar with the cutting slot.

31. The method of claim 30, wherein a ratio of the second thickness divided by the width of the at least one cutting slot is at least 0.8.

32. The method of claim 30, wherein the bone is at least one of a metatarsal and a cuneiform.

33. The method of claim 30, wherein positioning the cutting slot of the bone cutting guide over the bone to be cut comprises positioning a first cutting slot of the bone cutting guide over a first bone to be cut and positioning a second cutting slot of the bone cutting guide over a second bone to be cut, the first bone and the second bone being separated by a joint.
